(19) United States
(12) Patent Application Publication
(10) Pub. No.: US 2005/0034510 A1
(43) Pub. Date: Feb. 17, 2005

(54) Coolant service monitoring system

(76) Inventor: Charlie Bun Han, Saddle Brook, NJ (US)

Correspondence Address: Charlie B. Han, 37255 HANCOCK STREET, CLINTON TOWNSHIP, MI, 48036-2549, US

(21) Appl. No.: 10/638,462

(22) Filed: Aug. 12, 2003

(51) Int. Cl. G01N 33/00; G01N 31/00; G01N 27/27

(52) U.S. Cl. 73/61.43; 422/82.01; 436/163; 340/603

(57) ABSTRACT

Engine coolant and its related components is a vital fluid in any internal combustion engine, however it is one of the most neglected systems in the vehicle. The newest domestic automobiles do not have service indicators for engine coolant; which would allow the owner to service the fluid before any serious damage occurs from electrolysis and metal breakdown. The proposed Coolant Service Monitoring System (CSMS) will utilize sensors to monitor the pH of the coolant mixture, as well as the DC voltage of the coolant with respect to ground. By monitoring the pH and voltage, the sensors can notify the operator when unsafe coolant conditions are present. Ultimately, by preventing excessive corrosion and accelerated electrolysis, automobile owners can save hundreds if not thousands of dollars by not having to replace heater cores, expensive radiators, and water pumps with the CSMS implemented in the vehicle.

### BACKGROUND OF THE INVENTION

The Coolant Service Monitoring System (CSMS) was designed to solve the mystery of knowing when to service the coolant in an automobile's cooling system. However, seeing how it could be applied to any engine that required coolant in the form of water and any type of anti-freeze, it would benefit a large amount of people. The guesswork involved in knowing to service the coolant would be reduced to an indicator in your instrument panel lighting up; notifying the operator that the coolant required replacement or servicing.

### BRIEF SUMMARY OF THE INVENTION

The Coolant Service Monitoring System (CSMS) is a two-stage device. It measures the pH (−log [H⁺] or the measurement of the concentration of the H⁺ ions) and the voltage drop between the coolant and common electrical ground. With engine cooling systems, a certain pH level would indicate the coolant is ready to be serviced and/or replaced. In addition, a high voltage reading (varies depending on radiator construction and coolant material) would indicate an advanced level of electrolysis. These readings would be processed through simple gauges or indicators notifying the operator if coolant service is required. The actual pH and voltage “critical” values vary depending on the material of the radiator as well as the additive package used in anti-freezes and coolants.

### DRAWING DIRECTIONS

FIG. 1:

FIG. 1 shows how the pH probe is inserted through a coolant passage and takes readings of the coolant. The data is then sent to a gauge and activates the gauge or readout if the readings exceed a certain value.

FIG. 2:

FIG. 2 is similar to FIG. 1 but shows the voltage sensor that is inserted through a similar coolant passage. The data can then be sent to a similar gauge and activates the gauge or readout if the readings exceed a certain value.

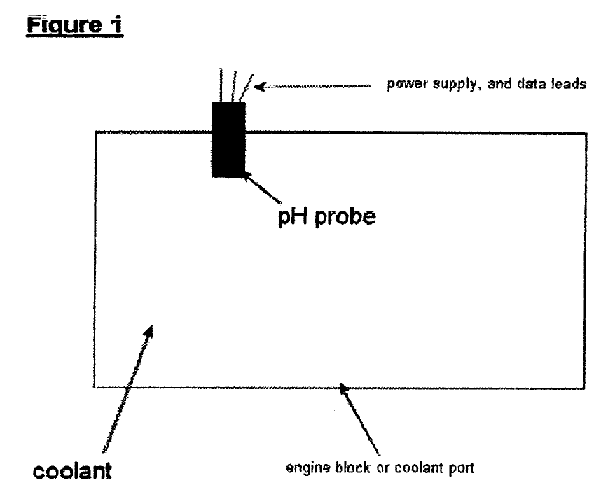

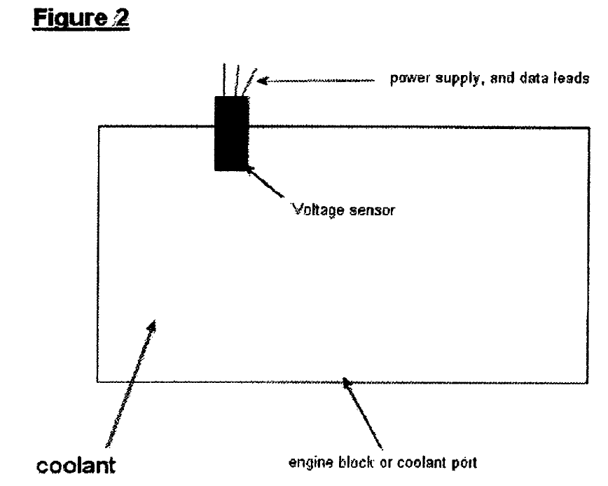

### DETAILED DESCRIPTION OF THE INVENTION

The Coolant Service Monitoring System (CSMS) is expected to be a low-cost coolant indicator system, so it is expected that the CSMS will employ inexpensive gauges or indicators that notify the operator of the engine if the coolant requires servicing.

The CSMS uses both a pH probe and a voltage sensor to take periodic readings of the engine coolant to determine if the coolant requires servicing. Since there are different radiator designs and coolant additive packages developed by various engine and automotive manufacturers, it is impossible to pinpoint exact critical pH and voltage readings. This is best left to the engine and automotive manufacturers.

The CSMS sensors are installed in areas of the engine that see a high volume of coolant flow in order to get the most updated readings available. The coolant pH and voltage is best sampled when the coolant is circulating.

The CSMS pH sensor is powered by any standard voltage source. In an automotive application, it may be preferred to directly use a +12V DC source such as the one from the car electrical system. The CSMS would take readings of the coolant and if the pH or voltage exceeds a pre-determined level (established by the engine or automotive manufacturer), the indicator or gauge will notify the operator that the coolant requires servicing.

In a standard ethylene glycol and water coolant system, a voltage above 0.4V DC requires attention because this indicates an advanced level of electrolysis and can accelerate corrosion of metal cooling containers. In a standard ethylene glycol and water coolant system, a pH lower than 8 demands immediate attention.

As a comparison, a fresh mixture of 50/50 ethylene glycol-based antifreeze and distilled water will show a voltage reading of 20 mV DC (with respect to common ground) or lower, while the pH will read 10.5 or higher.

The CSMS would take readings of the voltage and pH. If it reaches a critical value, such as a pH of 8.0 and a voltage of 0.25V DC, the indicator gauges or lights will then notify the operator that a coolant change is required or to service their vehicle at the nearest authorized dealership.

1). I claim that my Coolant Service Monitoring System will effectively monitor the condition of coolant based on pH and/or pOH levels and voltage drop with respect to common ground to ascertain the level of electrolysis and coolant breakdown for internal combustion engines and will warn the operator if any cooling system or coolant service action is required through direct interfaces with gauges or indicator lights, and/or data bus interface.

2). I claim that with the correct pH and/or pOH sensor systems selected for optimized detection of pH levels ranging from 1 to 14 in accordance with the cooling system's original equipment manufacturer specifications, and with a voltage sensor optimized to detect a critical value between 0 Volts and 250 Volts, the Coolant Service Monitoring System will be a cost effective solution to effectively reduce warranty claims related to cooling system corrosion.
